(19) United States
(12) Patent Application Publication
(10) Pub. No.: US 2026/0286274 A1
(43) Pub. Date: Sep. 24, 2026

(54) BIOPROCESSING SYSTEM AND METHOD AND COMPONENTS THEREOF

(71) Applicant: Pall Life Sciences Belgium BV, Hoegaarden (BE)

(72) Inventor: Benjamin Conradt, Waterloo (BE)

(73) Assignee: Pall Life Sciences Belgium BV, Hoegaarden (BE)

(21) Appl. No.: 19/085,221

(22) Filed: Mar. 20, 2025

(51) Int. Cl. C12M 1/34; C12M 1/00; C12M 1/06

(52) U.S. Cl. CPC C12M 41/36; C12M 23/26; C12M 27/02; C12M 29/16; C12M 41/26

(57) ABSTRACT

A bioprocessing vessel is provided that includes a vessel interior, a biomass holder, and one or more sensors. The vessel interior is configured to receive a fluid for bioprocessing. The biomass holder is located within the vessel interior. The biomass holder surrounds an impeller. The biomass holder includes a first end and a second end opposite the first end in a vertical direction. The biomass holder is configured to be flexible in the vertical direction. The one or more sensors are configured to extend between the first end and the second end. The one or more sensors have a vertical length in the vertical direction that is greater than a horizontal length in a horizontal direction.

### BACKGROUND

### Technical Field

Examples of the subject matter herein relate generally to equipment used to manufacture chemical agents, particularly biopharmaceuticals, as well as monitoring methods thereof. The equipment may include disposable containers.

### Discussion of Art

Mixers and bioreactors are often employed to carry out biochemical and biological processes and/or manipulate liquids and other products of such processes. Many bioreactors utilize closed, single use vessels, e.g., rigid, semi-rigid, or flexible containers and bags, that are fitted to or mounted on reusable equipment. Such vessels may also be referred to in the art as a single use systems (SUS). Such vessels may be in the form of rigid single use vessels, such as those used with fixed-bed bioreactors, as well as flexible bags that are housed within and are supported by an outer rigid structure such as a stainless-steel tank.

In fixed-bed reactors, the single use vessel is generally a rigid structure or housing that include a bed of carriers, e.g., polymeric fibers, which creates a large surface area for cells to adhere to. In use, the vessel may be fitted to an open platform or housing which may, for example, provide heat and include a drive unit for an impeller located withing the rigid single use vessel. Oxygenated culture medium is circulated through the bed to facilitate cell growth.

In these systems, various sensors are utilized to measure characteristics of the products to ensure the products are within desired parameters. The vessels and the various sensors may have a large footprint and may be rigid. The large foodprint and rigid material may be associated with increased material quantity and cost. Additionally, the size of the vessel, and components thereof, may increase the stress put on the vessel and in turn create quality concerns and difficulty in manufacturing.

The dimensions of single use vessel may vary somewhat from vessel to vessel due to manufacturing tolerances, e.g., variance due to material shrinkage, etc. This may be particularly true with rigid single use vessels such as those used with fixed bed systems. Given these potential variances, decreasing size and increasing flexibility may reduce material and cost and increase reliability and longevity.

It may be desirable to have improvements in design and methods of use of bioprocessing systems in order reduce the size and increase the reliability. It may be desirable to have a system and method that differs from those that are currently available.

### BRIEF DESCRIPTION

In accordance with an aspect of the invention, a bioprocessing vessel is described. The bioprocessing vessel includes a vessel interior, a biomass holder, and one or more sensors. The vessel interior is configured to receive a fluid for bioprocessing. The biomass holder is located with the vessel interior and may surround an impeller. The biomass holder has a first end and a second end opposite the first end in a vertical direction. The biomass holder is configured to be flexible in the vertical direction. The one or more sensors are configured to extend between the first end and the second end. The one or more sensors have a vertical length in the vertical direction that is greater than a horizontal length in a horizontal direction.

In an embodiment, the biomass holder may be bellow shaped. In one example, the one or more sensors may include a temperature sensor. In one example, the one or more sensors may include a pH sensor. In yet another embodiment, the one or more sensor may include a cell density sensor.

In one embodiment, the biomass holder may be configured to be flexible responsive to internal pressure from the bioprocessing vessel. In one example, a sensor holder may be provided and may be configured to contain one of the one or more sensors. In one example, the sensor holder may be rigid.

In accordance with another aspect of the invention, a bioprocessing system is described. The bioprocessing system includes a bioprocessing vessel and a housing. The bioprocessing vessel includes an impeller and a biomass holder. In one example, the biomass holder may surround the impeller. The biomass holder has a first end and a second end opposite the first end in a vertical direction. The first end and the second end are connected by a flexible material. The flexible material is configured to be compressible in the vertical direction. The bioprocessing vessel also has one or more sensors configured to extend between the first end and the second end. The one or more sensors have a vertical length in the vertical direction that is greater than a horizontal length in a horizontal direction. The housing has an interior configured to facilitate bioprocessing of a fluid in the bioprocessing vessel. The housing is in operative contact with the bioprocessing vessel during the use of the bioprocessing system.

In an embodiment, the flexible material may include convolute tubing. In one example, the biomass holder may be bellow shaped.

In one embodiment, the one or more sensors may include a biomass sensor. In one example, the vertical length of the one or more sensors may be at least twice the horizontal length of the one or more sensors.

In one embodiment, a sensor holder may be configured to contain one of the one or more sensors.

In accordance with yet another aspect of the invention, a method of operating a bioreactor is provided. The method includes adding a fluid to a bioprocessing vessel having an impeller and a biomass holder having a flexible material extending in a vertical direction between a first end and a second end opposite the first end. The flexible material is configured to be compressible in the vertical direction. The method includes sensing one or more characteristics of the fluid using a biomass sensor. The biomass sensor is configured to extend between the first end and the second end. The biomass sensor includes a vertical length in the vertical direction that is greater than a horizontal length in a horizontal direction.

In an embodiment, the biomass holder may be bellow shaped. In one example, the method may include providing convolute tubing with the flexible material.

In one embodiment, the vertical length of the biomass sensor is at least twice the horizontal length of the biomass sensor. The flexible material may be configured to be flexible responsive to internal pressure from the bioreactor.

In one embodiment, the method may further include providing a sensor holder to contain the biomass sensor. In one example, the sensor holder may be rigid.

### BRIEF DESCRIPTION OF THE DRAWINGS

The subject matter may be understood from reading the following description of non-limiting examples, with reference to the attached drawings, wherein below:

FIG. 1 is a perspective view of a bioprocessing device, according to an embodiment of the invention;

FIG. 2 is a cross-sectional view of a vessel of a bioprocessing device, according to an embodiment of the invention;

FIG. 3 is a front view of a biomass holder of a bioprocessing device, according to an embodiment of the invention;

FIG. 4 is a portion of a cross-sectional view of a vessel and biomass holder of a bioprocessing device, according to an embodiment of the invention; and

FIG. 5 is a top view of a vessel and a sensor of a bioprocessing device.

### DETAILED DESCRIPTION

Unless defined otherwise, all technical and scientific terms used herein have the same meaning as commonly understood by one of ordinary skill in the art to which this invention pertains.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioprocessing vessels having a wall or a portion of a wall that is flexible, single-use flexible bags, single-use rigid fixed bed containers, other containers or conduits commonly used in biological or chemical processing, including, for example, cell culture/purification systems, fermentation systems, mixing systems, media/buffer preparation systems, and filtration/purification systems.

Embodiments may be utilized in connection with a wide variety of biological and biochemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. “Bioprocessing device” as used herein, includes bioreactor, mixer, fermenter, support structures, e.g., rigid tanks and the like, as well as a single-use reactor vessels, such as a rigid fixed-bed reactor vessel. Certain embodiments may be suitable for use in other industries where precise, easy to use levelling is desirable.

While embodiments are shown and described as for use with fixed-bed bioreactors, embodiments are not so limited. Certain embodiments may be suitable for use with stirred tank bioreactors and mixers as well as various other bioprocessing devices. Embodiments are likewise not limited to any specific type of viral vector or cell line.

The bioprocessing systems may be related to the manufacture of chemical and/or biological products, for example, biopharmaceuticals. The bioprocessing system may utilize disposable containers. The use of single use containers or disposable containers in manufacturing processes has increased significantly in recent years. Reducing the size, and thus the material required for such containers, presents an opportunity for a more cost effective and efficient system.

Given the distance components of the bioprocessing assembly may travel and the precision needed during assembly, damage during transit and assembly is a real concern. By reducing the size of the components and the whole bioprocessing assembly, the risk of damage is reduced, and the cost of transportation is also reduced.

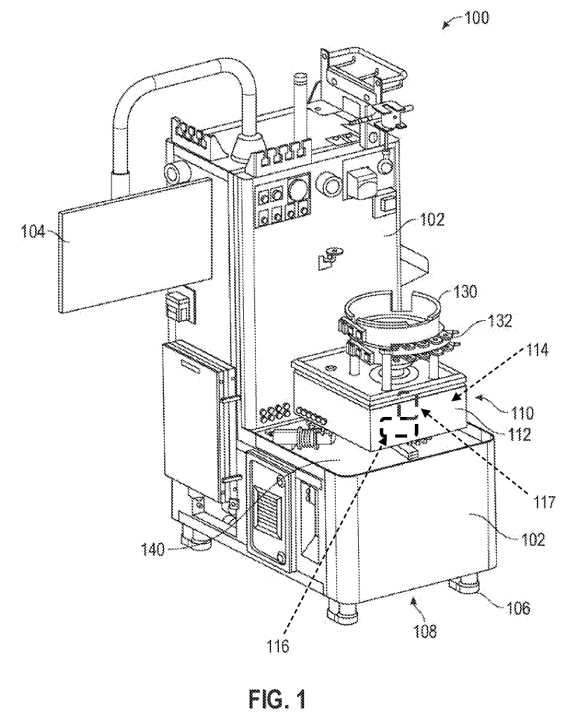

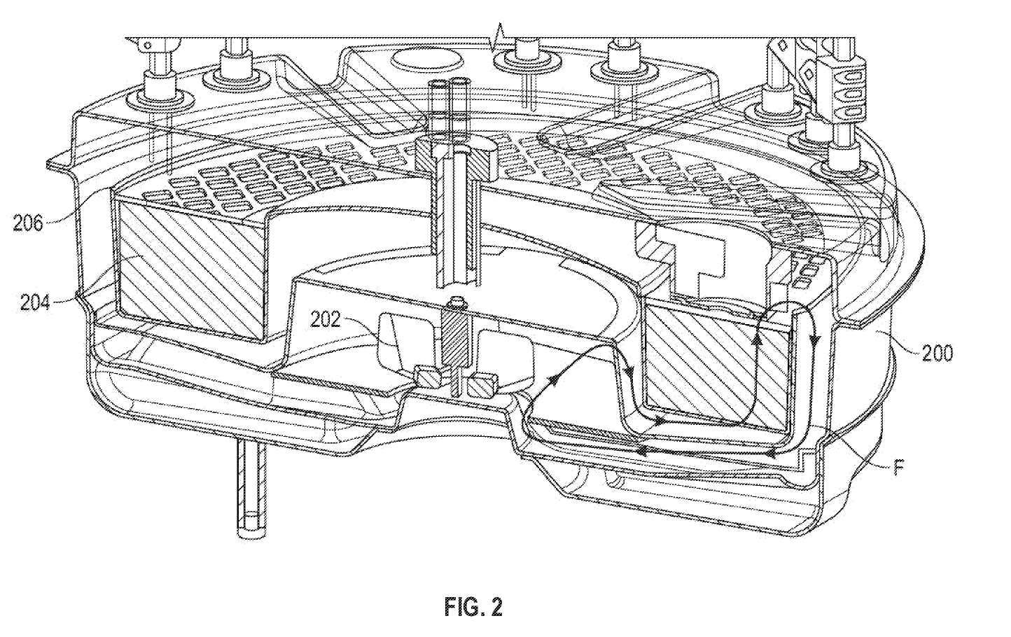

FIGS. 1 and 2 illustrate a bioprocessing device 100, and components thereof, suitable for use with embodiments of the present invention. The bioprocessing device 100 is a fixed-bed bioreactor that includes, among other components, a control assembly 102, which, as shown, is a substantially L-shaped, rigid cabinet. The control assembly 102 has an interior (not shown) that houses, for example, electrical panels, actuators, and fluid-flow/process automation related equipment. The control assembly 102 also includes a user interface 104 and a plurality of legs 106, which, as will be appreciated, contact the floor 108 beneath the bioprocessing device 100.

The bioprocessing device 100 further includes an interface apparatus 110 which is configured to interface with a vessel 200, which is a single-use fixed bed vessel. The interface apparatus 110 includes a housing 112 having an interior 114 that is configured to facilitate bioprocessing of a fluid in the vessel 200. To that end, the interior 114 of the housing 112 has a drive motor 116, that is operatively connected to a gearbox 117, which is in turn operatively connected to a magnetic shaft/drive head. In embodiments, the interior 114 of the housing 112 may be selectively opened and closed via a door and may be sealed via a gasket/seal. In use, a drive head may be magnetically coupled to an impeller 202 within the single use vessel 200 such that rotation of the drive head rotates the impeller 202 within the vessel 200 to agitate fluid therein.

In an embodiment, the housing 112 is configured to receive and/or support a vessel support receptacle 130 that, in turn, is configured to receive the vessel 200. In particular, the vessel support receptacle 130 may provide heat to the vessel 200 in addition to receiving and/or supporting the vessel 200. In embodiments, the receptacle 130 itself may be single use, and, to that end, may be manufactured from a rigid polymeric material. In other embodiments, the vessel support receptacle 130 may be integral to the vessel 200 or omitted. The housing 112 may further include a tubing management mechanism 132 which may be a U-shaped bracket having a plurality of teeth between which tubes and or valves may be placed.

As will be appreciated, while the depicted fixed bed bioreactor may be particularly suitable for embodiments of the invention, embodiments are not limited to any specific configuration of fixed bed reactor and may be used with various reactors. Embodiments may be suitable for bench scale reactors as well as larger production scale models. Certain embodiments may be suitable for use with reactors having a fixed-bed volume of .2 to 25L or more.

Turning back to FIGS. 1 and 2, in use, the vessel 200 may be placed within the vessel support receptacle 130. Fluid input and output lines are attached and the vessel 200 is configured for fluid processing. Referring to FIG. 2, the vessel 200 may include a fixed bed 204, which creates a large surface area for cells to adhere to. Importantly, aerated culture media is circulated through the fixed bed 204 via the impeller 202. In particular, aerated media is circulated through the fixed bed 204 along a flow path F.

After passing through the fixed bed 204, the media overflows the outer circumferential wall 206 of the fixed bed 204 creating a waterfall-like falling film. This generates high rates of oxygen transfer and CO2 stripping, allowing for the maintenance of high cell densities.

The fixed bed design of the system is well-suited for industrialization of adherent cell processes and virus production for both gene therapy and vaccines, providing a large cell growth surface area, while keeping the footprint small. Additionally, the fixed bed design enables high specific productivity.

As previously mentioned, however, the dimensions of single use vessels may vary somewhat due to stacked manufacturing tolerances and floor levelling of the structure to which a single use vessel is attached may not be ideal or sufficient to compensate. It may be advantageous to have a smaller vessel due to the reduced materials needed, and thus reduced cost associated with manufacture.

The vessel 200 may contain one or more sensors. The sensors may be configured to monitor various characteristics of the vessel and the contents thereof. The sensors may measure temperature, pH, dissolved oxygen (DO), pressure, cell count, biomass, weight, volume, or the like. The sensors may continuously measure critical culture conditions and cell density. The sensors may be pre-installed with the vessel 200. In another embodiment, the sensors may be installed in the vessel prior to use and may be removed after use.

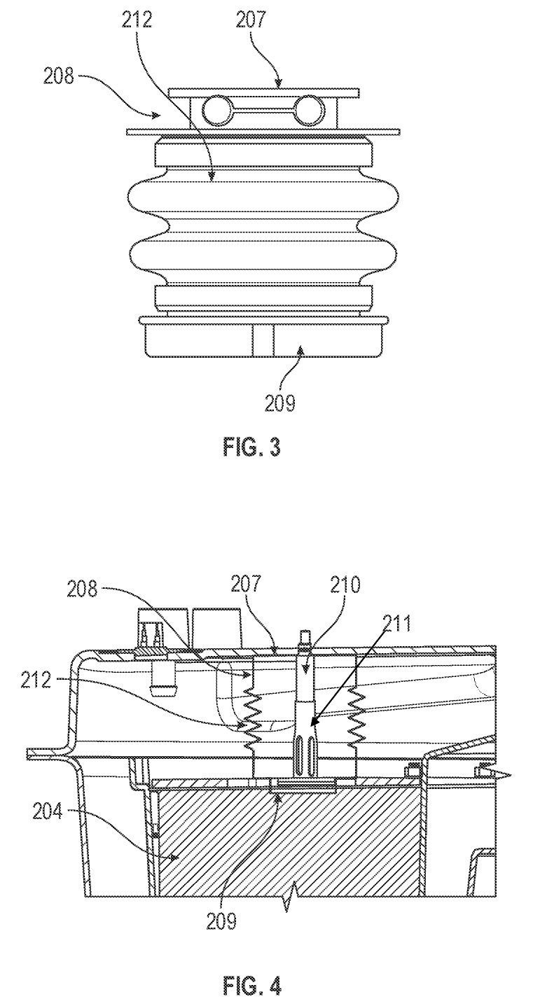

The vessel 200 may include a biomass holder 208 located within the vessel interior. FIG. 3 illustrates a side view of a biomass holder 208 according to one example. The biomass holder 208 and be positioned above the fixed bed 204, as illustrated in FIG. 4. However, in other embodiments, the biomass holder 208 may be positioned below the fixed bed 204 or laterally adjacent to the fixed bed 204.

The biomass holder 208 may be configured to contain a portion of the cells being processed. The biomass holder 208 may surround the impeller 202, and may extend from a first end 207 to a second end 209. The second end 209 is opposite the first end in a vertical direction. One or both of the first end 207 and the second end 209 may be connecting portions that connect the biomass holder 208 to a device, for example, to the fixed bed 204. The connecting portions may be removably connected to the device or may be fixedly connected to the device in other embodiments.

In one embodiment, the biomass holder 208 may be flexible in the vertical direction. The biomass holder 208 may be capable of flexing responsive to internal pressure from the vessel 200. The biomass holder 208 may include sidewall portions including bellow shaped portions 212. In one example, the sidewall portions of the biomass holder may include accordion shaped portions. However, in other examples, the biomass holder 208 may have a substantially linear sidewall portions that extends between the first end 207 and the second end 209. The biomass holder 208 may be made of an elastomeric material that is durable but permits flexibility of the biomass holder 208.

In one example, the first end 207 and the second end 209 may be a rigid material, while sidewall portions extending between the first end 207 and the second end may be a flexible material. In another embodiment, the sidewall portions between the first end 207 and the second end 209 may include a rigid portion and a flexible portion. The rigid portion of the sidewall portion may increase strength, durability, and longevity of the biomass holder, and the flexible portion of the sidewall portion may permit compression and flexibility to accommodate high stress and/or high pressure events within the bioprocessing system.

The flexible nature of the biomass holder 208 reduces the risk of internal stress and damages during high stress and/or pressure situations, for example during leak tight testing. In one example, the biomass holder 208 may include convolute tubing to allow flexibility and reduce stress and risk of damage when pressure in the bioreactor is high. The convolute tubing may be the sidewall portions of the biomass holder, in one example. The flexibility of the biomass holder 208 may reduce a stress on a top plate of the fixed bed 204, which may result in fewer quality concerns and easier manufacturability of the bioprocessing device.

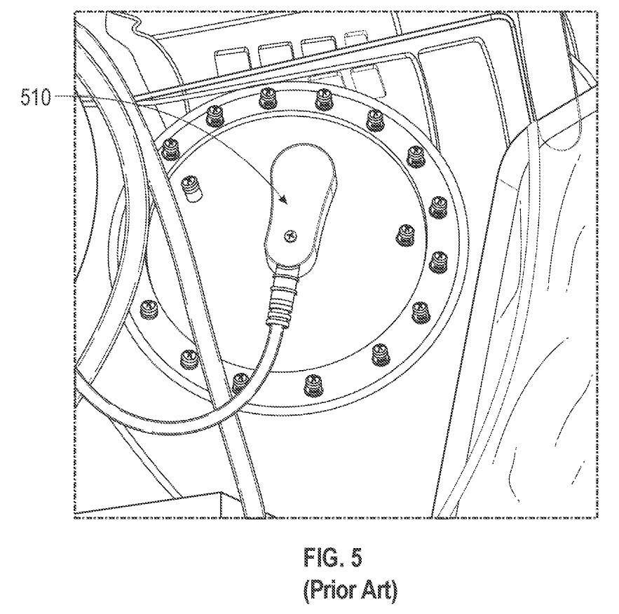

Additionally, the biomass holder 208 may include a sensor 210. The sensor 210 may be a biomass sensor. In typical bioprocessing devices, a sensor 510 may be positioned horizontally, as illustrated in prior art FIG. 5. However, in the present invention, the biomass sensor 210 may be arranged vertically, as shown in FIG. 4. By replacing a horizontal biomass sensor with a vertical biomass sensor, a diameter of the biomass holder may be reduced. As such, the material needed for manufacture may be reduced as well. Further, by using a vertical biomass sensor rather than a horizontal biomass sensor, the shape of the biomass holder may be modified. For example, the footprint may be reduced, as the biomass holder may be changed from a wider, larger footprint (e.g., donut shaped) to a narrower, cylindrical shape with a smaller footprint. The smaller footprint permits a more efficient arrangement that takes up less space.

In one example, a diameter of the biomass holder is reduced by moving from the horizontal biomass sensor to the vertical biomass sensor. The diameter may be reduced by between 15%-250% by changing from the horizontal to the vertical biomass sensor. The reduced diameter may additionally allow the biomass holder to be able to be integrated into a wider variety of bioprocessing systems. The size and dimensions of the biomass holder may be selected and/or modified based on the desired use characteristics and the size of the bioprocessing system into which it will be used. While this range is provided, the range is not meant to be limiting, but rather exemplary. The diameter may be reduced by less than or greater than the listed range.

Another benefit of the reduced footprint of the biomass holder is that it takes up less space when positioned on top of the fixed bed. By taking up less space on top of the fixed bed, there is a resulting decrease in obstacles for the media flowing through the system. The decreased obstacles may result in a more efficient processing of the media in the system.

The biomass holder may be removably coupled with the fixed bed. Said another way, the biomass holder may be selectively removed from the fixed bed for inspection, cleaning, maintenance, and the like. The fixed bed may contain a connection means to couple the biomass holder. In another example, the biomass holder may be fixedly coupled to the fixed bed.

In one embodiment, a length of the sensor 210 in the vertical direction is greater than a length of the sensor 210 in a horizontal direction. The length in the vertical direction may be twice the length in the horizontal direction. In other examples, the length in the vertical direction may be greater than twice the length in the horizontal direction. The length in the vertical direction may be selected based on the dimensions of the bioprocessing system for which the sensor will be used.

In one example, the sensor 210 may be container within a sensor holder 211. The sensor holder may be rigid and may be sized and positioned to be within the biomass holder of the bioprocessing device. The rigid sensor holder may protect the sensor. Specifically, where the biomass holder flexes in response to pressure, the rigid sensor holder may ensure that the biomass holder being flexed or compressed does not damage or impact the sensor within the biomass holder. The sensor holder may stabilize and protect the sensor from forces and pressures within the system.

In one embodiment, a bioprocessing vessel is described. The bioprocessing vessel includes a vessel interior, a biomass holder, and one or more sensors. The vessel interior is configured to receive a fluid for bioprocessing. The biomass holder is located with the vessel interior and may surround an impeller. The biomass holder has a first end and a second end opposite the first end in a vertical direction. The biomass holder is configured to be flexible in the vertical direction. The one or more sensors are configured to extend between the first end and the second end. The one or more sensors may have a vertical length in the vertical direction that may be greater than a horizontal length in a horizontal direction.

In one example, the biomass holder may be bellow shaped. In one example, the one or more sensors may include a temperature sensor. In one example, the one or more sensors may include a pH sensor. In one example, the one or more sensors may include a cell density sensor.

In one example, the biomass holder may be configured to be flexible responsive to internal pressure from the bioprocessing vessel. In one example, a sensor holder may be provided and may be configured to contain one of the one or more sensors. In one example, the sensor holder may be rigid.

In one embodiment, a bioprocessing system is described. The bioprocessing system includes a bioprocessing vessel and a housing. The bioprocessing vessel includes an impeller and a biomass holder surrounding the impeller. The biomass holder has a first end and a second end opposite the first end in a vertical direction. The first end and the second end are connected by a flexible material. The flexible material is configured to be compressible in the vertical direction. The bioprocessing vessel also has one or more sensors configured to extend between the first end and the second end. The one or more sensors have a vertical length in the vertical direction that is greater than a horizontal length in a horizontal direction. The housing has an interior configured to facilitate bioprocessing of a fluid in the bioprocessing vessel. The housing is in operative contact with the bioprocessing vessel during the use of the bioprocessing system.

In one example, the flexible material may include convolute tubing. In one example, the biomass holder may be bellow shaped.

In one example, the one or more sensors may include a biomass sensor. In one example, the vertical length of the one or more sensors may be at least twice the horizontal length of the one or more sensors.

In one example, a sensor holder may be configured to contain one of the one or more sensors.

In one embodiment, a method of operating a bioreactor is provided. The method includes adding a fluid to a bioprocessing vessel having an impeller and a biomass holder having a flexible material extending in a vertical direction between a first end and a second end opposite the first end. The flexible material is configured to be compressible in the vertical direction. The method includes sensing one or more characteristics of the fluid using a biomass sensor. The biomass sensor is configured to extend between the first end and the second end. The biomass sensor includes a vertical length in the vertical direction that is greater than a horizontal length in a horizontal direction.

In an example, the biomass holder may be bellow shaped. In one example, the method may include providing convolute tubing with the flexible material.

In one example, the vertical length of the biomass sensor is at least twice the horizontal length of the biomass sensor. The flexible material may be configured to be flexible responsive to internal pressure from the bioreactor.

In one example, the method may further include providing a sensor holder to contain the biomass sensor. In one example, the sensor holder may be rigid.

As used herein, an element or step recited in the singular and proceeded with the word “a” or “an” do not exclude the plural of said elements or operations, unless such exclusion is explicitly stated. Furthermore, references to “one embodiment” of the invention do not exclude the existence of additional embodiments that incorporate the recited features. Moreover, unless explicitly stated to the contrary, embodiments “comprising,” “comprises,” “including,” “includes,” “having,” or “has” an element or a plurality of elements having a particular property may include additional such elements not having that property. In the appended claims, the terms “including” and “in which” are used as the plain-English equivalents of the respective terms “comprising” and “wherein.” Moreover, in the following claims, the terms “first,” “second,” and “third,” etc. are used merely as labels, and do not impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function devoid of further structure.

The above description is illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein define the parameters of the inventive subject matter, they are exemplary embodiments. Other embodiments will be apparent to one of ordinary skill in the art upon reviewing the above description. The scope of the inventive subject matter should, therefore, be determined with reference to the appended claims, along with the full scope of equivalents to which such claims are entitled.

Use of phrases such as “one or more of … and,” “one or more of … or,” “at least one of …and,” and “at least one of … or” are meant to encompass including only a single one of the items used in connection with the phrase, at least one of each one of the items used in connection with the phrase, or multiple ones of any or each of the items used in connection with the phrase. For example, “one or more of A, B, and C,” “one or more of A, B, or C,” “at least one of A, B, and C,” and “at least one of A, B, or C” each can mean (1) at least one A, (2) at least one B, (3) at least one C, (4) at least one A and at least one B, (5) at least one A, at least one B, and at least one C, (6) at least one B and at least one C, or (7) at least one A and at least one C.

This written description uses examples to disclose several embodiments of the inventive subject matter, including the best mode, and to enable one of ordinary skill in the art to practice the embodiments of inventive subject matter, including making and using any devices or systems and performing any incorporated methods. The patentable scope of the inventive subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A bioprocessing vessel, comprising:
a vessel interior configured to receive a fluid for bioprocessing;
a biomass holder located within the vessel interiorthe biomass holder having a first end and a second end opposite the first end in a vertical direction, wherein the biomass holder is configured to be flexible in the vertical direction;
one or more sensors that are configured to extend between the first end and the second end, the one or more sensors having a vertical length in the vertical direction that is greater than a horizontal length in a horizonal direction.

2. The bioprocessing vessel of claim 1, wherein the biomass holder is bellow shaped.

3. The bioprocessing vessel of claim 1, wherein the one or more sensors include a cell density sensor.

4. The bioprocessing vessel of claim 1, wherein the one or more sensors include a pH sensor.

5. The bioprocessing vessel of claim 1, wherein the biomass holder is configured to be flexible responsive to internal pressure from the bioprocessing vessel.

6. The bioprocessing vessel of claim 1, further comprising a sensor holder configured to contain one of the one or more sensors.

7. The bioprocessing vessel of claim 1, wherein the biomass holder is configured to surround an impeller.

8. A bioprocessing system, comprising:
a bioprocessing vessel that includes an impeller and a biomass holder, the biomass holder having a first end and a second end opposite the first end in a vertical direction,
wherein the first end and the second end are connected by a flexible material, the flexible material configured to be compressible in the vertical direction, the bioprocessing vessel also having one or more sensors configured to extend between the first end and the second end, the one or more sensors having a vertical length in the vertical direction that is greater than a horizontal length in a horizonal direction; and
a housing having an interior configured to facilitate bioprocessing of a fluid in the bioprocessing vessel, the housing being in operative contact with the bioprocessing vessel during use of the bioprocessing system.

9. The bioprocessing system of claim 8, wherein the flexible material includes convolute tubing.

10. The bioprocessing system of claim 8, wherein the biomass holder is bellow shaped.

11. The bioprocessing system of claim 8, wherein the one or more sensors includes a biomass sensor configured to measure a cell density.

12. The bioprocessing system of claim 8, wherein the vertical length of the one or more sensors is at least twice the horizontal length of the one or more sensors.

13. The bioprocessing system of claim 8, further comprising a sensor holder configured to contain one of the one or more sensors.

14. A method of operating a bioreactor, comprising:
adding a fluid to a bioprocessing vessel having an impeller and a biomass holder having a flexible material extending in a vertical direction between a first end and a second end opposite the first end, wherein the flexible material is configured to be compressible in the vertical direction;
sensing one or more characteristics of the fluid using a biomass sensor, the biomass sensor configured to extend between the first end and the second end, wherein the biomass sensor includes a vertical length in the vertical direction that is greater than a horizontal length in a horizonal direction.

15. The method of claim 14, wherein the biomass holder is bellow shaped.

16. The method of claim 14, providing convolute tubing with the flexible material.

17. The method of claim 14, wherein the vertical length of the biomass sensor is at least twice the horizontal length of the biomass sensor.

18. The method of claim 14, wherein the flexible material is configured to be flexible responsive to internal pressure from the bioreactor.

19. The method of claim 14, further comprising providing a sensor holder to contain the biomass sensor configured to measure a cell density.

20. The method of claim 19, wherein the sensor holder is rigid.
